(19) United States
(12) Patent Application Publication
(10) Pub. No.: US 2005/0014817 A1
(43) Pub. Date: Jan. 20, 2005

(54) Fluorothiophene derivatives, process for preparing them and pharmaceutical compositions containing them

(75) Inventors: Delphine Compere, Paris (FR); Anne-Claude Dublanchet, Antony (FR); Karine Courte, Issy-les-Moulineaux (FR); Stephane Blais, Palaiseau (FR)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/884,066

(22) Filed: Jul. 2, 2004

Related U.S. Application Data
(60) Provisional application No. 60/494,676, filed on Aug. 12, 2003.

(30) Foreign Application Priority Data
Jul. 3, 2003 (EP) 03291642.1

(51) Int. Cl. A61K 31/381; C07D 333/32

(52) U.S. Cl. 514/444; 514/448; 549/64; 549/59

(57) ABSTRACT

The invention provides compounds of Formula (I), 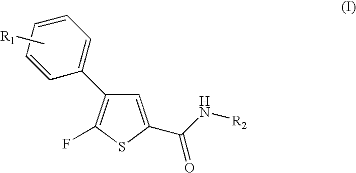 stereoisomers thereof, or pharmaceutically acceptable salts of said compounds or stereoisomers wherein R1 and R2 are as defined herein as well as compositions comprising the same, processes for making the same, and methods of using the same to treat a variety of diseases, including, those requiring interaction with metalloproteases, and more specifically with macrophage metalloelastase (MMP-12), and for the prevention and treatment of respiratory pathologies such as chronic obstructive bronchopneumopathy (COPD), emphysema, chronic bronchitis, chronic pulmonary inflammation, asthma, cystic fibrosis, acute respiratory distress syndrome (ARDS), respiratory allergies including allergic rhinitis, and also diseases associated with the production of TNFα including severe fibrotic pulmonary disease, pulmonary sarcoidosis and silicosis. The compounds of the present invention also show inhibitory activity on metalloprotease-13 (MMP-13), making them useful for the treatment of pathologies involving this enzyme, such as cancer, osteoporosis, osteoarthritis, arthritis, rheumatoid arthritis, atherosclerosis, multiple sclerosis and cardiac insufficiency.

### FIELD OF THE INVENTION

The present invention relates to fluorothiophene derivatives for interacting with metalloproteases, and more specifically with macrophage metalloelastase (MMP-12), and for the prevention and treatment of respiratory pathologies such as chronic obstructive bronchopneumopathy (COPD), emphysema, chronic bronchitis, chronic pulmonary inflammation, asthma, cystic fibrosis, acute respiratory distress syndrome (ARDS), respiratory allergies including allergic rhinitis, and also diseases associated with the production of TNFα including severe fibrotic pulmonary disease, pulmonary sarcoidosis and silicosis. The compounds of the present invention also show inhibitory activity on metalloprotease-13 (MMP-13), making them useful for the treatment of pathologies involving this enzyme, such as cancer, osteoporosis, osteoarthritis, arthritis, rheumatoid arthritis, atherosclerosis, multiple sclerosis and cardiac insufficiency. In addition, the invention relates to methods and processes concerning the same, and to compositions containing the same.

### BACKGROUND OF THE INVENTION

Metalloproteases (MMPs) are a large family of proteases that degrade the extracellular matrix and are secreted especially by mesenchymal cells, macrophages and polymorphonuclear leukocytes. Metalloproteases are classified into several subfamilies depending on their primary structure and their specificity. These families especially include collagenases (MMP-1, MMP-8 and MMP-13), stromelysins (MMP-3 and MMP-10), gelatinases (MMP-2 and MMP-9), matrilysin (MMP-7), macrophage metalloelastase (MMP-12) and also MMPs of membrane-bound type (MMP-14, MMP-15, MMP-16 and MMP-17).

MMPs are zinc metalloproteases that have the ability to degrade virtually all the components of the extracellular matrix, ie the interstitium and the basal membranes. Increased synthesis of these enzymes is found in many destructive diseases (inflammatory arthritis, atherosclerosis, tumoral invasion and angiogenesis). MMPs (in particular those with powerful elastolytic activity) are involved in the physiopathology of asthma and chronic obstructive bronchopneumopathies including tobacco-related pulmonary emphysema (COPD).

Human macrophage elastase (HME or MMP-12) shows all the characteristics of the other MMPs. It degrades many macromolecules of the extracellular matrix (gelatin, fibronectin and laminin) and especially elastin. MMP-12 is not synthesized by the circulating monocytes but solely by macrophages or monocytes differentiated in vitro into macrophages. The pathology of emphysema is characterized by destruction of the elastin present in the walls of the pulmonary alveolae. Demonstration of the increase in the level of MMP-12 during the manifestation of this pathology thus suggests a predominant role of this enzyme in the occurrence and development of this disease. Similarly, studies have demonstrated the absence of development of emphysema in MMP-12-deficient mice, these mice being exposed for a long time to cigarette smoke (Science 1997, 277, 2002-2004). More recently, also using MMP-12-deficient mice, in an asthma model, a group has suggested the involvement of MMP-12 in the development of chronic asthma (FASEB, 2002, 16, A590). These results clearly demonstrate that human macrophage elastase (MMP-12) inhibitors might be very useful for preventing and treating chronic respiratory pathologies such as chronic obstructive pulmonary bronchitis (COPD), emphysema, chronic bronchitis and chronic pulmonary inflammation, and also respiratory pathologies caused by an inflammation phenomenon, such as asthma, mucoviscidosis, acute respiratory distress syndrome (ARDS), repiratory allergies including allergic rhinitis and also diseases associated with the production of TNFα including severe fibrotic pulmonary disease, pulmonary sarcoidosis and silicosis.

All metalloproteases have a catalytic domain consisting of 162 to 173 amino acids containing the active site of the enzyme. A Zn²⁺ ion is present in the active site, to which it is bound via histidine residues. This site is one of the preferred points of attachment of synthetic MMP inhibitors, since it especially allows the creation of a stable, powerful chelation centre that is readily accessible to small molecules. Thus, all the powerful inhibitors described in the literature contain a chemical function such as a hydroxamic acid allowing chelation between the zinc atom of the catalytic site of the metalloprotease and the said inhibitor. This chelation ensures blockage of the active site and results in inhibition of the said enzyme.

One of the major problems of inhibition of this type is the absence of selectivity or the low degree of selectivity, since all MMPs contain a zinc ion in their active site. The second problem associated with these powerful but generally poorly selective inhibitors is the toxicity associated with the presence of a chemical function such as a hydroxamic acid.

One of the objects of the invention is thus to provide novel compounds that have inhibitory properties on type 12 metalloprotease (MMP-12). A solution has been found by producing novel fluorothiophene derivatives, and also by using the said compounds in pharmaceutical compositions that can be used in the prevention and treatment of pathologies associated with an inhibition of MMP-12.

Several scientific articles and patent applications describe compounds comprising a central thiophene unit. Among this literature, mention may be made of patent application WO 98/23605, which describes thien-2-ylcarboxamide derivatives substituted in position 4 with a cyclic system and in position 5 with a trifluoromethyl group. These compounds are claimed for their bactericidal and fungicidal activity. Patent application WO 96/16954 also describes compounds optionally comprising a 4-aryl-thien-2-ylcarboxamide system in which the amide function may be substituted with a phenyl group, which are useful for their antifungal properties.

Mention may also be made of patent application JP 63 175 853 or the article Chem. Commun. 2001, 8, 759-760, which describe compounds comprising a substituted thiophene group, these compounds constituting fluorescence photoregulators or photographic developers.

None of these documents describes or suggests for these compounds inhibitory activity on MMP-12 and a potential use of products of this type in the treatment of respiratory pathologies, which is a novel property of the compounds claimed by the Applicant. More specifically, none of these documents describes (5-fluoro-4-phenyl)thien-2-yl carboxamide derivatives as MMP-12 inhibitors.

### SUMMARY OF THE INVENTION

The invention provides compounds of Formula (I), 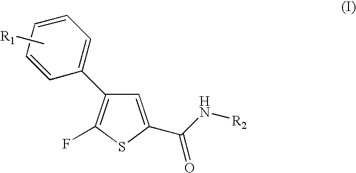
stereoisomers thereof, or pharmaceutically acceptable salts of said compounds or stereoisomers wherein R1 and R2 are as defined below as well as compositions comprising the same, processes for making the same, and methods of using the same to treat a variety of diseases, including, those requiring interaction with metalloproteases, and more specifically with macrophage metalloelastase (MMP-12), and for the prevention and treatment of respiratory pathologies such as chronic obstructive bronchopneumopathy (COPD), emphysema, chronic bronchitis, chronic pulmonary inflammation, asthma, cystic fibrosis, acute respiratory distress syndrome (ARDS), respiratory allergies including allergic rhinitis, and also diseases associated with the production of TNFα including severe fibrotic pulmonary disease, pulmonary sarcoidosis and silicosis. The compounds of the present invention also show inhibitory activity on metalloprotease-13 (MMP-13), making them useful for the treatment of pathologies involving this enzyme, such as cancer, osteoporosis, osteoarthritis, arthritis, rheumatoid arthritis, atherosclerosis, multiple sclerosis and cardiac insufficiency.

### DETAILED DESCRIPTION

The invention provides compounds of Formula (I), 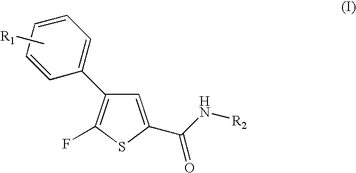
in which:
R₁ represents a group selected from halo(C₁-C₆)alkoxy, (C₁-C₆)alkyl, (C₁-C₆)alkoxy, (C₁-C₆)alkylthio and —R₃ in which:
R₃ represents a group selected from phenyl, cyclohexyl and a heterocycle, each of these groups being optionally substituted with one or two groups, which may be identical or different, selected independently of each other from halogen, halo(C₁-C₆)alkyl-, halo(C₁-C₆)alkoxy-, (C₁-C₆)alkyl, cyano, cyano(C₁-C₆)alkyl-, hydroxyl, (C₁-C₆)alkoxy, phenoxy, (C₁-C₆)alkyl-SO₂—, (C₁-C₆)alkylcarbonyl, benzoyl, hydroxy(C₁-C₆)alkyl-, and amino optionally substituted with one or two (C₁-C₆)alkyl groups which may be identical or different;
R₂ represents a group —U—R₄ in which:
U represents a linear (C₁-C₄)alkylene chain optionally substituted with a group selected from carboxyl, carboxy(C₁-C₆)alkyl-, (C₁-C₆)alkyloxycarbonyl, (C₁-C₆)alkyloxycarbonyl(C₁-C₆)alkyl-, aminocarbonyl and aminocarbonyl(C₁-C₆)alkyl in which the amino portion is optionally substituted with one or two (C₁-C₆)alkyl groups, which may be identical or different,
R₄ represents a phenyl, cyclohexyl or morpholin-4-yl group, each of these groups being optionally substituted with one or two groups, which may be identical or different, selected independently of each other from halogen, —OR₅, —CO₂R₅ and —W—CO₂R₅, in which:
R₅ represents a hydrogen atom or a (C₁-C₆)alkyl group,
W represents a linear or branched (C₁-C₆)alkylene chain, the isomers thereof and the addition salts thereof with a pharmaceutically acceptable acid or base.

The term “heterocyclic group” means a saturated, unsaturated or aromatic, 5- or 6-membered monocyclic system, comprising 1 or 2 hetero atoms, which may be identical or different, selected from oxygen and nitrogen: furyl, pyrrolyl, pyrazolyl, pyridyl, pyrimidyl, pyrazinyl, indolyl, quinolyl, isoquinolyl, imidazolyl, N-pyrrolidinyl, 3,6-dihydro-2H-pyrid-1-yl, etc.

The term “(C₁-C₆)alkyl” means a linear or branched carbon-based chain containing from 1 to 6 carbon atoms; as a guide, mention may be made of the following groups: methyl, ethyl, propyl, isopropyl, tert-butyl, neopentyl, hexyl, etc.

The term “(C₂-C₆)alkenyl group” means a linear or branched carbon-based chain containing from 2 to 6 carbon atoms and one or more double bonds; as a guide, mention may be made of the following groups: vinyl, allyl, 3-buten-1-yl, 2-methyl-buten-1-yl, hexenyl, etc.

The term “(C₁-C₆)alkoxy” means an alkyl group as defined above linked via an oxygen atom; as a guide, mention may be made of the following groups: methoxy, ethoxy, n-propyloxy, tert-butyloxy, etc.

The term “halo(C₁-C₆)alkyl” means a linear or branched carbon-based chain containing from 1 to 6 carbon atoms and substituted with 1 to 6 halogen atoms; as a guide, mention may be made of the following groups: trifluoromethyl, 2,2,2-trifluoroethyl, etc.

The term “halo(C₁-C₆)alkoxy” means a linear or branched carbon-based chain containing from 1 to 6 carbon atoms and substituted with 1 to 6 halogen atoms, the said chain being linked to the compound of formula (I) via an oxygen atom; as a guide, mention may be made of the following groups: trifluoromethoxy, 2,2,2-trifluoroethoxy, etc.

The term “halogen atom” means an atom selected from bromine, chlorine, fluorine and iodine.

The term optical isomers refer to the racemic mixtures, enantiomers and diastereoisomers.

According to one particularly advantageous variant of the invention, the phenyl group in the compounds of formula (I) is substituted with a group R₁ as defined in formula (I), located in the para position.

The groups R₁ that are preferred according to the invention are groups selected from trifluoromethoxy, 4-acetylphenyl, 4-pyridyl, 3-pyridyl, N-pyrrolydinyl, 1-methylpyrrol-3-yl, 3,6-dihydro-2H-pyrid-1-yl, 2-hydroxyphenyl and 2-hydroxy-4-pyridyl.

In a particularly advantageous manner, R₁ represents a group selected from trifluoromethoxy, 4-acetylphenyl and 4-pyridyl.

According to one preferred variant of the invention, R₂ represents a group —U—R₄ in which U represents a linear (C₁-C₂)alkylene chain and R₄ represents a cyclohexyl group substituted in position 4- with a carboxyl group. More specifically, the preferred compounds of the invention are those for which the said carboxyl group is of trans stereochemistry.

According to another preferred variant of the invention, R₂ represents a group —U—R₄ in which U represents a methylene group substituted with a carboxymethyl or aminocarbonylmethyl group and R₄ represents a phenyl group. In a particularly advantageous manner, the carbon atom bearing the group R₄ taking the definition phenyl is of (R) absolute configuration.

The isomers, and also the addition salts with a pharmaceutically acceptable acid or base, of the variants and the preferred compounds form an integral part of the invention.

The invention also relates to the pharmaceutically acceptable salts of the compounds of formula (I). A review of pharmaceutically acceptable salts is described especially in J. Pharm. Sci., 1977, 66, 1-19.

The expression “pharmaceutically acceptable acids” means non-toxic organic or mineral acids. Among the pharmaceutically acceptable acids that may be mentioned, without any limitation, are hydrochloric acid, hydrobromic acid, sulphuric acid, phosphonic acid, nitric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid, benzoic acid, toluenesulphonic acid, etc.

The expression “pharmaceutically acceptable bases” means non-toxic organic or mineral bases.

Pharmaceutically acceptable bases include sodium hydroxide, potassium hydroxide, lithium hydroxide, calcium hydroxide, triethylamine, tert-butylamine, 2-diethylaminoethanol, ethanolamine, ethylenediamine, dibenzylethylenediamine, piperidine, pyrrolidine, morpholine, piperazine, benzylamine, arginine, lysine, histidine, glucamine, glucosamine, quaternary ammonium hydroxides, etc.

In general, the expression “isomers of the compounds of the invention” means optical isomers such as enantiomers and diastereoisomers. More particularly, the pure enantiomeric forms of the compounds of the invention may be separated from mixtures of enantiomers that are reacted with a releasable agent for resolving the racemic mixtures, the said agent itself existing in the form of a pure enantiomer, allowing the corresponding diastereoisomers to be obtained. These diastereoisomers are then separated according to the separation techniques that are well known to those skilled in the art, such as crystallization or chromatography, and the resolving agent is then removed using the standard techniques of organic chemistry, to produce a pure enantiomer. In another manner, the pure enantiomeric forms of the compounds of the invention may be separated by chromatography on a chiral column.

The terms “treating”, “treated”, or “treatment” as employed herein includes prevantative (e.g., prophylactic), palliative, or curative uses or results.

The compounds of the invention that are present in the form of a mixture of diastereoisomers are isolated in pure form by using standard separation techniques such as chromatographies.

In certain particular cases, the process for separating the compounds of the invention may lead to the predominant formation of one enantiomer or one diastereoisomer relative to the other.

The invention also covers the process for preparing the compounds of formula (I). More particularly, the compounds of formula (I) may be obtained from the compounds of formula (II): 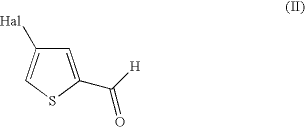
in which Hal represents a halogen atom,
which compounds of formula (II) are:
   either reacted, under basic palladium coupling conditions, with a compound of formula (III): 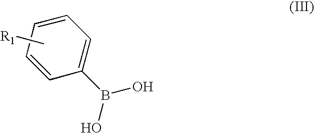
      in which R₁ is as defined in formula (I),
to give the compounds of formula (IV): 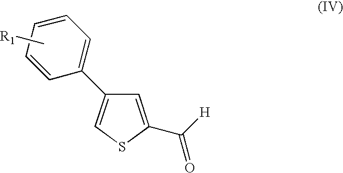
   in which R₁ is as defined above,
which compounds of formula (IV) are subjected to oxidation conditions in the presence, for example, of silver nitrate in a basic and polar medium, to give the compounds of formula (V): 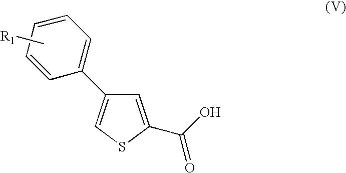
   in which R₁ is as defined above,
which compounds of formula (V) are treated, under peptide coupling conditions in the presence, for example, of a coupling agent and in a basic medium, with a compound of formula (VI):
   H₂N—R₂ (VI)
   in which R₂ is as defined in formula (I):
to give the compounds of formula (VII): 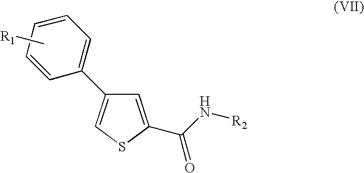
   in which R₁ and R₂ are as defined above,
which compounds of formula (VII) are then treated with 1-chloromethyl-4-fluoro-1,4-diazoniabicyclo[2,2,2]octane bis(tetrafluoroborate) in the presence of acetonitrile, to give the compounds of formula (I): 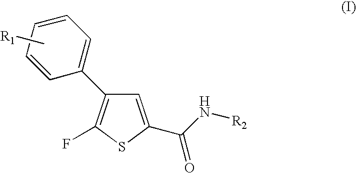
   in which R₁ and R₂ are as defined above,
which compounds of formula (I) may optionally undergo a basic hydrolysis in the case where R₂ represents a group U—R₄ in which U represents a linear (C₁-C₄)alkylene chain substituted with a group selected from (C₁-C₆)alkyloxycarbonyl- and (C₁-C₆)alkyloxycarbonylalkyl-, to give the carboxylic acid equivalents of formula (I/a), a particular case of the compounds of formula (I): 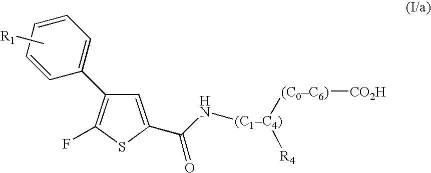
   in which R₁ and R₄ are as defined in formula (I),
which compounds of formula (I/a) may then be treated in a first step with oxalyl chloride, and then in a second step with an aqueous ammonia solution, or a primary or secondary amine, to give the compounds of formula (I/b), a particular case of the compounds of formula (I): 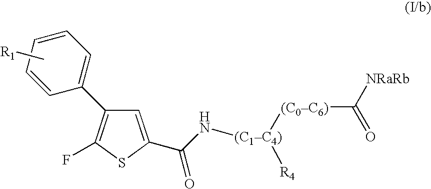
   in which R₁ and R₄ are as defined above, and Ra and Rb each represent a hydrogen atom or a (C₁-C₆)alkyl group,
   or reacted, after oxidation of the aldehyde function, optionally followed by an esterification of the carboxylic acid function obtained, with a phenylboronic acid that is suitably substituted, such as with a nitro group, to give, after two steps of standard synthesis for substitution of a nitro group with a halogen atom, the compounds of formula (VIII): 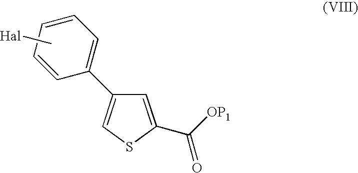
      in which P₁ represents a hydrogen atom or a linear or branched (C₁-C₆)alkyl group and Hal represents a halogen atom,
which compounds of formula (VIII) are then treated with 1-chloromethyl-4-fluoro-1,4-diazoniabicyclo[2,2,2]octane bis(tetrafluoroborate) in the presence of acetonitrile, to give the compounds of formula (IX): 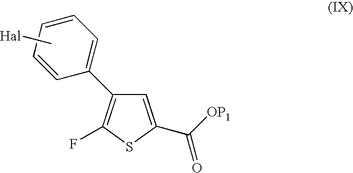
   in which P₁ and Hal are as defined above,
which compounds of formula (IX) are then reacted, under basic conditions and in the presence of a palladium catalyst, with a compound of formula (X): 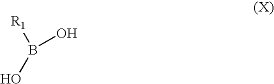
   in which R₁ is as defined in formula (I),
to give the compounds of formula (XII): 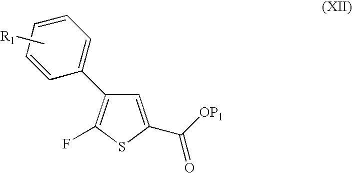
   in which R₁ and P₁ are as defined above,
which compounds of formula (XII) are treated, under peptide coupling conditions in the presence, for example, of a coupling agent and in a basic medium, with a compound of formula (VI) as defined above, also to give the compounds of formula (I): 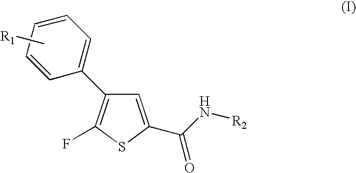
   in which R₁ and R₂ are as defined above,
compounds of formula (I), which are purified, where appropriate, according to a standard purification technique, which may, if so desired, be separated into the various isomers thereof according to a standard separation technique, and which are converted, where appropriate, into the addition salts thereof with a pharmaceutically acceptable acid or base.

According to one variant of the process of the invention, the fluorination in position 5 of the thienyl nucleus may be performed directly on the thienyl substituted in position 2 with a carbaldehyde function.

More particularly the compounds of formula (I) may be obtained from the compounds of formula (II): 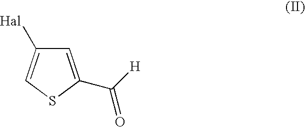
in which Hal represents a halogen atom,
which compounds of formula (II) are reacted, under basic palladium coupling conditions, with a compound of formula (III): 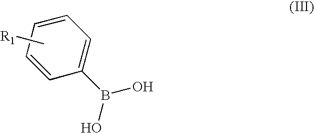
   in which R₁ is as defined in formula (I),
to give the compounds of formula (IVA): 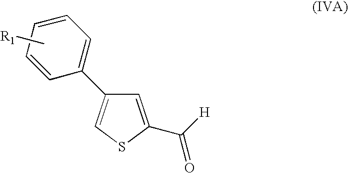
   in which R₁ is as defined above,
which compounds of formula (IVA) are then treated for 12 hours at 70° C. with 1.95 equivalents of 1-chloromethyl-4-fluoro-1,4-diazoniabicyclo[2,2,2]octane bis(tetrafluoroborate) in the presence of acetonitrile, to give the compounds of formula (IVB): 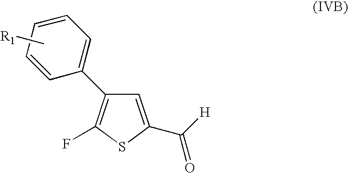
   in which R₁ is as defined above,
which compounds of formula (IVB) are then subjected to oxidation conditions conventionally used in organic synthesis, to give the compounds of formula (IVC): 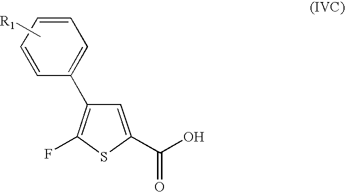
   in which R₁ is as defined above,
which compounds of formula (UVC) can then be treated in the same manner as the compounds of formula (XII) as described in the previous process, also to give the compounds of formula (I).

On account of their pharmacological properties as MMP-12 inhibitors, the compounds of the present invention are useful in the prevention and treatment of respiratory pathologies such as chronic obstructive bronchopneumopathy (COPD), emphysema, chronic bronchitis, chronic pulmonary inflammation, asthma, mucoviscidosis, acute respiratory distress syndrome (ARDS), respiratory allergies including allergic rhinitis, and also diseases associated with the production of TNFα, including severe fibrotic pulmonary diseases, pulmonary sarcoidosis and silicosis. The compounds of the present invention also show, to a lesser extent, inhibitory activity on metalloprotease-13 (MMP-13), making them potentially useful for the treatment of pathologies involving this enzyme, such as cancer, osteoporosis, osteoarthritis, arthritis, rheumatoid arthritis, atherosclerosis, multiple sclerosis and cardiac insufficiency.

Advantageously, the compounds of the present invention are useful for preventing and treating chronic obstructive bronchopneumopathy, emphysema and chronic bronchitis.

More particularly, the compounds of the present invention are useful for treating tobacco-related emphysema.

According to one variant of the invention, the compounds of formula (I) are useful for preventing and treating asthma.

The subject of the present invention is also pharmaceutical compositions containing as active principle at least one compound of formula (I), an isomer thereof or an addition salt thereof with a pharmaceutically acceptable acid or base, alone or in combination with one or more inert, non-toxic, pharmaceutically acceptable excipients or vehicles.

Among the pharmaceutical compositions according to the invention, mention will be made more particularly of those that are suitable for oral, parenteral (intravenous, intramuscular or subcutaneous), percutaneous or transcutaneous, intravaginal, rectal, nasal, perlingual or respiratory administration.

The pharmaceutical compositions according to the invention for parenteral injections especially comprise aqueous and non-aqueous sterile solutions, dispersions, suspensions or emulsions and also sterile powders to reconstitute injectable solutions or dispersions.

The pharmaceutical compositions according to the invention for solid oral administration especially comprise simple or sugar-coated tablets, sublingual tablets, sachets, gel capsules and granules, and, for oral, nasal or buccal liquid administration, especially comprise emulsions, solutions, suspensions, drops, syrups and aerosols.

The pharmaceutical compositions according to the invention for administration via the respiratory route especially comprise compositions in the form of solutions for aerosols or powders for inhalers. When the compositions are aerosols, for the use of liquid aerosols, the compositions may be stable sterile solutions or solid compositions dissolved at the time of use in apyrogenic sterile water, in physiological saline or in any other pharmaceutically acceptable vehicle. For use in the form of dry aerosols intended to be inhaled directly, the active principle is optionally finely divided or micronized, and combined with an inert, solid, water-soluble diluent or vehicle.

The pharmaceutical compositions for rectal administration are preferably suppositories, and those for percutaneous or transcutaneous administration especially comprise powders, aerosols, creams, ointments, gels and patches.

The pharmaceutical compositions mentioned above illustrate the invention but do not limit it in any way.

Among the inert, non-toxic, pharmaceutically acceptable excipients or vehicles that may be mentioned, as a guide and with no limitation, are diluents, solvents, preserving agents, wetting agents, emulsifiers, dispersants, binders, swelling agents, crumbling agents, retardants, lubricants, absorbing agents, suspension agents, colorants, flavourings, etc.

The practical dosage varies according to the age and weight of the patient, the route of administration, the pharmaceutical composition used, the nature and severity of the complaint, and the possible taking of associated treatments. The dosage ranges from 1 mg to 1000 mg in one or more dosage intakes per day.

The examples that follow illustrate the invention but do not limit it in any way.

The starting materials used are commercial products or products prepared according to known procedures from commercial compounds or compounds known to those skilled in the art. The various preparations give synthetic intermediates that are useful for preparing the compounds of the invention.

The structures of the compounds described in the examples and in the preparations were determined according to the usual spectrophotometric techniques (infrared (IR), nuclear magnetic resonance (NMR), mass spectrometry (MS) including electron spray (ES) mass spectrometry, etc.) and the purity was determined by high performance liquid chromatography (HPLC).

Abbreviations used in the procedures:

Selectfluor™: 1-chloromethyl-4-fluoro-1,4-diazoniabicyclo-[2,2,2]octane bis(tetrafluoroborate);
TOTU: O-[(ethoxycarbonyl)cyanomethylamino]-N-N-N′-N′-tetramethyluronium fluoroborate;
DME: 1,2-dimethoxyethane (or ethylene glycol dimethyl ether)
TFA: trifluoroacetic acid
HATU:O-(7-azabenzotriazol-1-yl)-N,N,N′,N′-tetramethyluronium hexafluorophosphate
tBME: tert-butyl methyl ether
   Preparation 1: Ethyl trans 4-(aminomethyl)cyclohexanecarboxylate 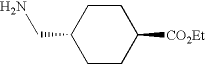
   0.4 ml of sulphuric acid is added to a solution of 0.2 g of trans-4-(aminomethyl)cyclohexanecarboxylic acid in 5 ml of ethanol. The reaction medium is refluxed for 17 hours and then concentrated under reduced pressure. The residue is taken up in ethyl acetate. The solution is basified to pH 9 by adding aqueous 1.0M sodium hydroxide solution, washed with water, dried over sodium sulphate, filtered and then concentrated under reduced pressure to give 0.129 g of a yellow oil corresponding to the expected product.

Yield: 55%

MS: MH⁺ 186

### EXAMPLE 1

### 4-({[5-Fluoro-4-(4-trifluoromethoxyphenyl)thien-2-yl]carboxamido}-methyl)cyclohexanecarboxylic Acid

Stage 1: 4-[4-(Trifluoromethoxy)phenyl]thiophene-2-carbaldehyde

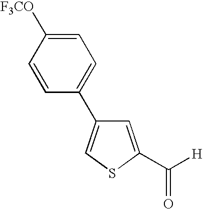

84.9 ml (2.1 equivalents) of 2.0M potassium phosphate solution and 2.8 g (0.03 equivalent) of tetrakis(triphenylphosphine)palladium(0) are added to a solution of 12.3 g of 4-bromothiophene-2-carbaldehyde and 20.0 g of 4-(trifluoromethoxy)phenylboronic acid (1.2 equivalents) in 70 ml of degassed DME. The reaction medium is stirred for 3 hours at 80° C. and then concentrated under reduced pressure. The residue obtained is taken up in ethyl acetate. The solution is then filtered through Celite, washed with water, dried over sodium sulphate, filtered and then concentrated under reduced pressure. Chromatography of the residue on silica gel (9/1 cyclohexane/ethyle acetate) allows 15.05 g of the expected product to be isolated.

Yield: 68%

¹H NMR (CDCl₃) δ (ppm): 10.0 (s, 1H), 8.0 (s, 1H), 7.80 (s, 1H), 7.55 (m,2H), 7.25 (m, 2H)
Stage 2: 4-[4-(Trifluoromethoxy)phenyl]thiophene-2-carboxylic Acid

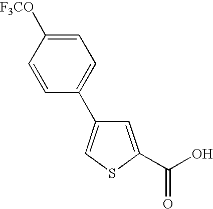

37.6 g (4 equivalents) of silver nitrate and 44.2 ml (8 equivalents) of aqueous 1.0M sodium hydroxide solution are added to a solution of 15.05 g of the compound obtained in stage 1 in 200 ml of ethanol. The reaction medium is stirred for 2 hours at 40° C. and then filtered through Celite and concentrated under reduced pressure. The aqueous phase is washed with 1.0M hydrochloric acid solution, extracted with ethyl acetate, dried over sodium sulphate, filtered and concentrated under reduced pressure, to give 15.51 g of a beige-coloured powder corresponding to the expected product.

Yield: 97.4%

MS: MH⁻ 287
Stage 3: Ethyl trans-4-({([4-(4-trifluoromethoxyphenyl)thien-2-yl]carboxamido}-methyl)cyclohexanecarboxylate

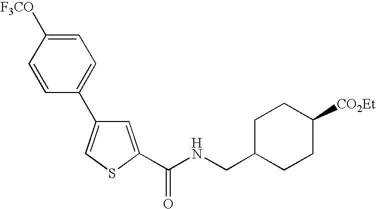

1.15 g of the product of Preparation 1 and 1.7 g of TOTU are successively added to a solution of 1.5 g of the compound obtained in stage 2 in 15 ml of anhydrous dichloromethane. The reaction medium is cooled to 0° C. and 1.8 ml of N,N-diisopropylethylamine are then added dropwise. After stirring overnight at room temperature, the reaction medium is hydrolysed and extracted with dichloromethane. The organic phases are combined, dried over sodium sulphate, filtered and evaporated under reduced pressure to give the expected product (2.124 g) after chromatography on silica gel eluted with a cyclohexane/ethyl acetate gradient (95:5 to 80:20 of 5 at 5%).

Yield: 90%

¹H NMR (CDCl₃) δ (Ppm): 7.70 (s, 1H), 7.60 (m, 2H), 7.50 (s, 1H), 6.0 (bs, 1H), 4.10 (q, 2H), 3.40 (t, 2H), 2.25 (m, 1H), 2.0 (m, 2H), 1.90 (m, 2H), 1.60 (m, 1H), 1.40 (m, 2H), 1.25 (t, 3H), 1.10 (q, 2H)

HPLC:95%
Stage 4: Ethyl trans-4-({([5-fluoro-4-(4-trifluoromethoxyphenyl)thien-2-yl]carboxamido}methyl)cyclohexanecarboxylate

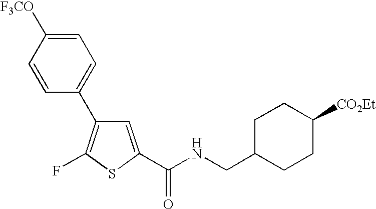

207 mg (1 equivalent) of Selectfluor™ are added to a solution of 265 mg of the compound obtained in stage 3 in 3.5 ml of acetonitrile. The reaction medium is stirred for 10 minutes at 70° C. and then for 3 hours at 50° C. The crude reaction mixture is hydrolysed and extracted with dichloromethane, and the combined organic phases are then washed with saturated sodium hydrogen carbonate solution, dried over magnesium sulphate, filtered and finally concentrated under reduced pressure to give 218 mg of a mixture corresponding to the expected fluoro product contaminated with the starting material (proportion 1:1 determined by HPLC).
Stage 5: trans 4-({([5-Fluoro-4-(4-trifluoromethoxyphenyl)thien-2-yl]carboxamido}-methyl)cyclohexanecarboxylic Acid

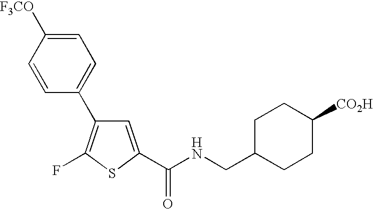

54 mg of lithium hydroxide (5 equivalents) are added to a solution of 213 mg of the mixture obtained in stage 4 above in 5 ml of an ethanol/water mixture (1/1). The reaction medium is stirred for 4 hours at 50° C. and then concentrated under reduced pressure. The solid obtained is taken up in water and acidified with 1.0M hydrochloric acid solution to pH 1. The precipitate formed is then filtered off, washed successively with water and with ether and then dried overnight, thus giving 149 mg of mixture. A reverse-phase chromatography (conditions column: C18. 21*50 mm; mode: gradient A) H₂O+0.1% TFA and B) acetonitrile+0.1% TFA from 30% of B to 95% over 13 min; flow rate: 30 ml/min; wavelength: 214 nm) allows 25 mg of the expected product to be isolated.

Yield (stages 4 and 5): 10%

¹H NMR (DMSO) δ (ppm): 11.99 (bs, 1H), 8.54 (t, 1H), 7.90 (d,1H), 7.73 (d, 2H), 7.51 (d, 2H), 3.11 (t, 2H), 2.14 (t, 1H), 1.90 (d, 2H), 1.78 (d, 2H), 1.48 (m, 1H), 1.25 (m, 2H), 0.98 (q, 2H)

HPLC: 97.92%

MS: MH⁺ 446

### EXAMPLE 2

### trans4-({[5-Fluoro-4-(4-[4-acetylphenyl]phenyl)thien-2-yl]carboxamido}methyl)cyclohexanecarboxylic Acid

Stage 1: Methyl 4-(4-nitrophenyl)thiophene-2-carboxylate

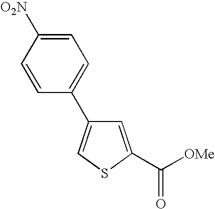

(4-Nitrophenyl)boronic acid (1.2 equivalents), tetrakis(triphenylphosphine)palladium(0) (0.03 equivalent) and 2.0M potassium phosphate solution (2.1 equivalents) are added to a solution under nitrogen of methyl 4-bromothiophene-2-carboxylate in 3.0 ml of degassed DME. The reaction medium is then stirred for 3 hours at 80° C., diluted with ethyl acetate (20 ml), washed with water (2×15 ml), dried over sodium sulphate, filtered and concentrated under reduced pressure. Chromatography of the residue on silica gel (98/2 dichloromethane/methanol) allows 1.94 g of the expected product to be isolated.

Yield: 78%

¹H NMR (CDCl₃) δ (ppm): 3.92 (s, 3H), 7.75 (d, 2H), 7.82 (s, 1H), 8.12 (s, 1H), 8.30 (d, 2H)
Stage 2: Methyl 4-(4-aminophenyl)thiophene-2-carboxylate

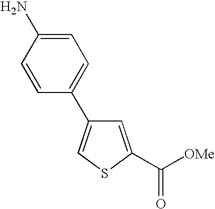

A solution of 1.94 g of the compound obtained in stage 1 above in 20 ml of methanol containing 194 mg of 10% palladium-on-charcoal is stirred under hydrogen pressure (10 bar). The reaction medium is then filtered through Celite and concentrated under reduced pressure to give 1.51 g of the desired product.

Yield: 88%

¹H NMR (DMSO) δ (ppm): 3.82 (s, 3H), 5.22 (s, 2H), 6.60 (d, 2H), 7.42 (d, 2H), 7.90 (s, 1H), 8.05 (s, 1H)

MS: MH⁺ 234
Stage 3: Methyl 4-(4-bromophenyl)thiophene-2-carboxylate

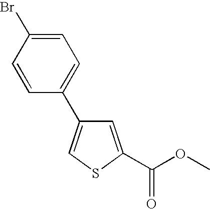

0.6 ml of concentrated hydrobromic acid is added to a solution of 103 mg of the product obtained in stage 2 above in 1.5 ml of water. The reaction medium is cooled to 0° C. and a solution of 35.5 mg of sodium nitrite (1.1 equivalents) in 0.5 ml of water is then added dropwise. After stirring for 1 hour at 0° C., a solution of 68 mg of copper bromide in 0.5 ml of concentrated hydrobromic acid is added dropwise. The reaction medium is stirred for a further 1 hour at 0° C. and then diluted with ethyl acetate (30 ml), washed successively with water (3×15 ml), with saturated sodium hydrogen carbonate solution (15 ml) and then with water (15 ml). The organic phase is dried over sodium sulphate, filtered and then concentrated under reduced pressure. Chromatography of the residue on silica gel (95/5 cyclohexane/ethyl acetate) allows 52 mg of the desired product to be isolated.

Yield: 40%

¹H NMR (CDCl₃) δ (Ppm): 8.05 (s, 1H), 7.65 (s, 1H), 7.55 (d, 2H), 7.45 (d, 2H), 3.92 (s, 3H)

HPLC: 91.4%
Stage 4: Methyl 4-(4-bromophenyl)-5-fluorothiophene-2-carboxylate

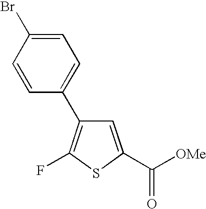

5.95 g of Selectfluor™ (1 equivalent) are added to a solution of 5 g of the compound obtained in stage 3 above in 50 ml of acetonitrile. The reaction medium is stirred for 17 hours at 70° C. After cooling to room temperature, the precipitate formed is filtered off and washed with water. The solid thus obtained corresponds to the fluoro derivative contminated with the starting material (60/40 HPLC proportion in favour of the expected product). The mixture is purified by successive chromatographies on silica gel eluted with an isocratic heptane/dichloromethane mixture (1:1) to give the expected product (2.05 g).

Yield: 40%

¹H NMR (DMSO) δ (ppm): 8.02 (d, 1H), 7.67 (m, 4H), 3.85 (s, 3H)

HPLC: 100%
Stage 5: Methyl 4-(4′-acetylbiphenyl-4-yl)-5-fluorothiophene-2-carboxylate

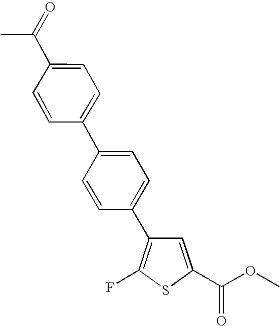

The product (310 mg) is obtained according to the process of stage 1 of Example 1, using 4-acetylphenylboronic acid as co-substrate.

Yield: 37%

¹H NMR (DMSO) δ (ppm): 8.05 (m,3H), 7.87 (m, 6H), 3.87 (s, 3H), 2.65 (s, 3H)

HPLC: 98%
Stage 6: 4-(4′-Acetylbiphenyl-4-yl)-5-fluorothiophene-2-carboxylic Acid

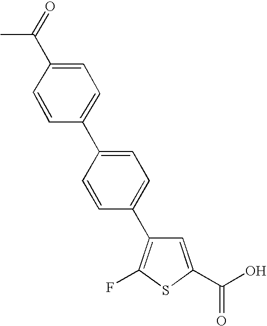

The product (230 mg) is obtained according to the process of stage 5 of Example 1, using the product obtained in the preceding stage as substrate.

Yield: 77%

¹H NMR (DMSO) δ (ppm): 8.05 (m, 3H), 7.85 (m, 6H), 6.0 (s, 3H)

HPLC: 80%
Stage 7: Ethyl trans-4-({[5-Fluoro-4-(4-[4-acetylphenyl]phenyl)thien-2-yl]carboxamido}methyl)cyclohexanecarboxylate

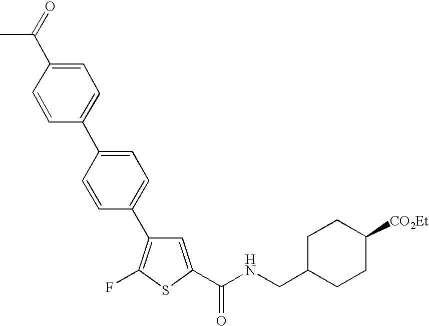

255 mg of HATU (1 equivalent) and then 149 mg of the product of Preparation 1 (1.2 equivalents) and 0.2 ml of N-ethyl-N,N-diisopropylamine (2 equivalents) are added to a solution of 228 mg of the above compound in 15 ml of anhydrous dimethylformamide. The reaction medium is stirred for 3 hours at room temperature and then hydrolysed. The precipitate formed is filtered off, washed with water and then dried. Chromatography of the solid obtained on silica gel (9/1 dichloromethane/ethanol) allows the expected product to be isolated (100 mg).

Yield: 29%

¹H NMR (DMSO) δ (ppm): 8.45 (t, 1H), 8.05 (m, 3H), 7.90 (m, 4H), 7.75 (d, 2H), 4.0 (m, 2H), 3.10 (t, 2H), 2.25 (t, 1H), 1.90 (d, 2H), 1.80 (d, 2H), 1.50 (m, 1H), 1.30 (q, 2H), 1.15 (m, 3H), 1.0 (q, 2H)

HPLC: 76.0
Stage 8: trans-4-({[5-Fluoro-4-(4-[4-acetylphenyl]phenyl)thien-2-yl]carboxamido}methyl)cyclohexanecarboxylic Acid

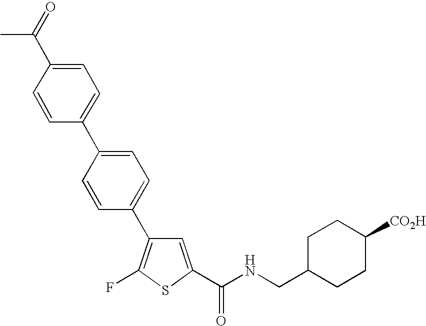

The product (21 mg) is obtained according to the process of stage 5 of Example 1, using the product obtained in the preceding stage as substrate.

Yield: 47%

¹H NMR (DMSO) δ (ppm): 12.0 (bs, 1H), 8.55 (t, 1H), 8.05 (d, 2H), 7.90 (d, 4H), 7.75 (d, 2H), 3.10 (t, 2H), 2.65 (s, 3H), 2.15 (t, 1H), 1.92 (d, 2H), 1.80 (d, 2H), 1.45 (m, 1H), 1.30 (q, 2H), 1.0 (q, 2H)

HPLC: 86.6%

### EXAMPLE 3

### 3-(R)-Phenyl-3-({5-fluoro-4-[4-(4-acetylphenyl)phenyl]thien-2 yl}carboxamido)-propanoic Acid

Stage 1: Ethyl 3-(R)-phenyl-3-([5-fluoro-4-[4-(4-acetylphenyl)phenyl]thien-2-yl]-carboxamido)propanoate

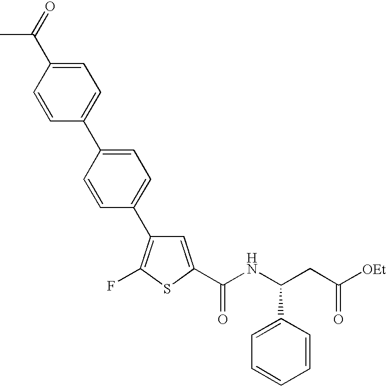

The product (70 mg) is obtained according to the process of stage 7 of Example 2, using ethyl (R)-3-amino-3-phenylpropanoate hydrochloride as co-substrate.

Yield (not optimized): 7%

HPLC: 98.0%

MS: MH⁺ 516
Stage 2: 3-(R)-Phenyl-3-([5-fluoro-4-[4-(4-acetylphenyl)phenyl]thien-2-yl]-carboxamido)propanoic Acid

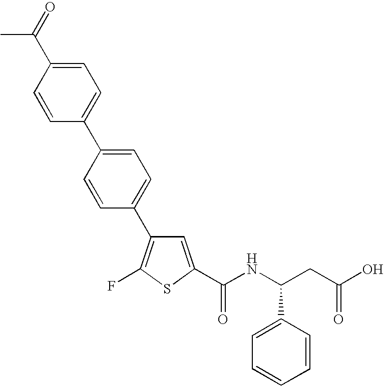

The product (25 mg) is obtained according to the process of stage 5 of Example 1 using the product obtained in the above stage as substrate.

Yield: 38%

¹H NMR (DMSO) δ (ppm): 12.41 (bs, 1H), 9.04 (bs, 1H), 8.14 (bs, 1H), 8.06 (d, 2H), 7.91 (t, 4H), 7.89 (d, 2H), 7.35 (m, 5H), 5.40 (dd, 1H), 2.84 (m, 2H), 2.62 (s, 3H)

HPLC: 98.32%

### EXAMPLE 4

### 3-(R)-Phenyl-3-({5-fluoro-4-[4-trifluoromethoxy)phenyl]thien-2-yl}carboxamido)propanoic Acid

Stage 1: 5-Fluoro-4-[4-(trifluoromethoxy)phenyl]thiophen-2-carbaldehyde

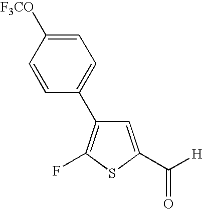

12.7 g (1.95 equivalents) of Selectfluor™ are added, under nitrogen, to a solution of 5 g of 4-[4-(trifluoromethoxy)phenyl]thiophene-2-carboxaldehyde obtained in stage 1 of Example 1 in 140 ml of acetonitrile. The reaction mixture is heated at 70° C. for 16 hours and then cooled and concentrated under reduced pressure. The residue, taken up in 50 ml of tBME, is hydrolysed and extracted several times with tBME. The combined organic phases are dried over magnesium sulphate, filtered and evaporated under vacuum to give a brown oil, which is purified on silica gel (98/2 heptane/tBME). The desired fractions are combined to give 1.8 g of product in the form of a white powder.

Yield: 30%

¹H NMR (DMSO) δ (ppm): 9.88 (s, 1H), 8.35 (s, 1H), 7.82 (d, 2H), 7.50 (d, 2H)
Stage 2: 5-Fluoro-4-[4-(trifluoromethoxy)phenyl]thiophen-2-yl-carboxylic acid

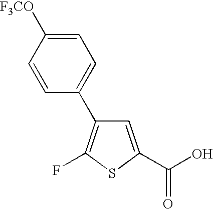

The product (900 mg) is obtained according to the process of stage 2 of Example 1, using the product obtained in the above stage as substrate.

Yield: 47%

¹H NMR (DMSO) δ (ppm): 13.50 (bs, 1H), 7.93 (s, 1H), 7.82 (d, 2H), 7.50 (d, 2H)
Stage 3: Ethyl 3-(R)-phenyl-3-([5-fluoro-4-[4-(trifluoromethoxy)phenyl]thien-2-yl]-carboxamido)propanoate

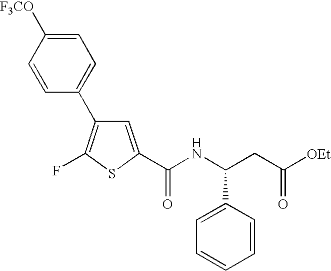

The product (1.054 g) is obtained according to the process of stage 7 of Example 2, using ethyl (R)-3-amino-3-phenylpropanoate hydrochloride as co-substrate.

Yield: 84%

HPLC: 99.0%

MS: MH⁺ 482, MH⁻ 480
Stage 4: 3-(R)-Phenyl-3-([5-fluoro-4-[4-(trifluoromethoxy)phenyl]thien-2-yl]carboxamido)propanoic Acid

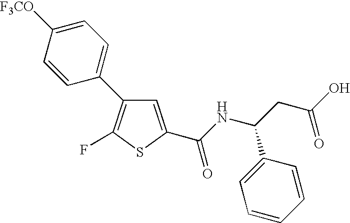

The product (771 mg) is obtained according to the process of stage 5 of Example 1, using the product obtained in stage 3 above as substrate.

Yield: 77%

¹H NMR (DMSO) δ (ppm): 12.34 (bs, 1H), 8.98 (d, 1H), 8.06 (s, 1H), 7.74 (d, 2H), 7.51 (d, 2H), 7.36 (m, 4H), 7.26 (m, 1H), 5.38 (m, 1H), 2.83 (m, 2H)

HPLC: 98.68%

MS: MH⁻ 452

### EXAMPLE 5

### 3-(R)-Phenyl-3-({5-fluoro-4-[4-(4-acetylphenyl)phenyl]thien-2-yl}carboxamido)propanamide

Stage 1: 3-(R)-Phenyl-3-([5-fluoro-4-[4-trifluoromethoxyphenyl]thien-2-yl]carboxamido)propanoyl Chloride

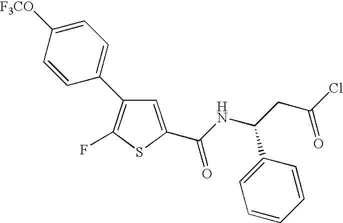

2.54 ml (4 equivalents) of oxalyl chloride are added dropwise, at room temperature, to a solution of 681 mg of the compound obtained in stage 4 of Example 4 in 10 ml of anhydrous dichloromethane containing a few drops of dimethylformamide. After reaction for 45 minutes, the reaction medium is concentrated under reduced pressure to give a yellow oil corresponding to the expected product, which is used in crude form in the following stage.
Stage 2: 3-(R)-Phenyl-3-({5-fluoro-4-[4-trifluoromethoxyphenyl]thien-2-yl}carboxamido)propanamide

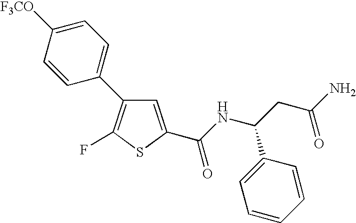

A large excess of 28% aqueous ammonia solution is added at 0° C. to the solution obtained in stage 1 above taken up in 10 ml of anhydrous tetrahydrofuran. The ice bath is removed and the mixture is stirred overnight at room temperature. The crude reaction mixture is concentrated under reduced pressure, hydrolysed and extracted with dichloromethane. The organic phases are combined, dried over sodium sulphate, filtered and concentrated under reduced pressure to give a residue, which is purified by chromatography on silica gel (80:20 cyclohexane/ethyl acetate). The desired product (146 mg) is obtained in the form of a white powder.

Yield: 21.5%

¹H NMR (DMSO) δ (ppm): 8.93 (d, 1H), 8.06 (d, 1H), 7.74 (d, 2H), 7.52 (d, 2H), 7.35 (m, 5H), 7.24 (t, 1H), 6.85 (s, 1H), 5.41 (q, 1H), 2.66 (m, 2H)

HPLC: 99.8%

MS: MH⁺ 453, MH⁻ 451

### EXAMPLE 6

### (R)-3-Phenyl-3-({5-fluoro-4-[4-(4-pyridyl)phenyl]thien-2-yl}-carboxamido)propanoic Acid

Stage 1: Methyl [4-(4-pyridyl)phenyl]-5-fluorothiophene-2-carboxylate

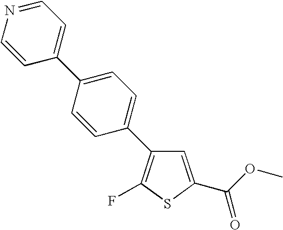

The product (260 mg) is obtained according to the process of stage 1 of Example 1, using 4-pyridylboronic acid as co-substrate.

Yield: 34%

HPLC: 98.69%

MS: MH⁺ 314
Stage 2: [4-(4-Pyridyl)phenyl]-5-fluorothiophene-2-carboxylic Acid Hydrochloride

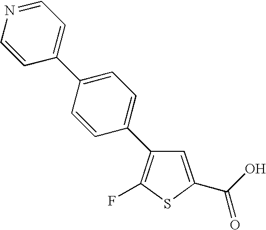

The product (223 mg) is obtained in the form of a salt according to the process of stage 5 of Example 1, using the product obtained in the above stage as substrate.

Yield: 80%

HPLC: 98.28%

MS: MH⁺ 300, MH⁻ 298
Stage 3: Ethyl 3-(R)-phenyl-3-([5-fluoro-4-[4-(4-pyridyl)phenyl]thien-2-yl]carboxamido)propanoate

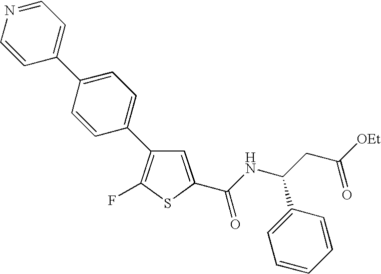

The product (220 mg) is obtained according to the process of stage 7 of Example 2, using ethyl (R)-3-amino-3-phenylpropanoate hydrochloride as co-substrate.

Yield: 70%

HPLC: 95.07%

MS: MH⁺ 475, MH⁻ 473
Stage 4: 3-(R)-Phenyl-3-([5-fluoro-4-[4-(4-pyridyl)-phenyl]thien-2-yl]carboxamido)propanoic Acid Hydrochloride

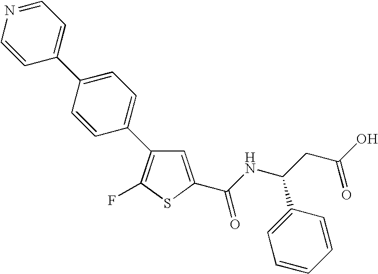

The product (166 mg) is obtained in the form of a salt according to the process of stage 5 of Example 1, using the product obtained in the above stage as substrate.

Yield: 75%

¹H NMR (DMSO) δ (ppm): 12.25 (bs, 1H), 9.18 (d, 1H), 8.94 (d, 2H), 8.37 (d, 2H), 8.31 (s, 1H), 8.17 (d, 2H), 7.87 (d, 2H), 7.44 (d, 2H), 7.35 (t, 2H), 7.26 (t, 1H), 5.40 (q, 1H), 2.88 (m, 2H)

HPLC: 98.56%

MS: MH⁺ 447

### EXAMPLE 7

### Pharmacological Studies on the Compounds of the Invention

In Vitro Evaluation of the Inhibitory Activity of the Compounds of the Invention on MMP-12:

The inhibitory activity of the compounds of formula (I) on metalloprotease-12 is evaluated by testing the capacity of the compounds of the invention to inhibit the proteolysis of a peptide that is an MMP-12 substrate.

The substrate peptide used (fluorigenic peptide-1: FP-1) in the test has the following sequence: Mca-Pro-Leu-Gly-Leu-Dap(Dnp)-Ala-Arg-NH₂.

The inhibitory activity of a compound of formula (I) is expressed as the IC₅₀ value, which represents the concentration of inhibitor for which a 50% inhibition of the metalloprotease is observed.

The reaction starts with the sequential addition of 41 μl of FP-1 substrate (final concentration of 10 μM) to a buffer solution of 50 mM of Tris-HCl and 10 mM of CaCl₂, and containing 5 mM of hydroxamic acid and 5 μl of the enzyme diluted in a 0.005% Brij-35 buffer solution. The microplates are incubated for 20 minutes at room temperature.

The compounds of the invention are tested at concentrations ranging from 0.3 to 30 μM.

The measurement of the amount of proteolysis of the peptide substrate is monitored by means of a measurement of absorbance at 405 nm using a microplate spectrophotometer, at room temperature. The IC₅₀ values are calculated from curves in which the percentage of the catalytic activity relative to the control is represented on the x-axis and the inhibitor concentration is represented on the y-axis.

The test described above for the inhibition of MMP-12 is adapted and used to determine the capacity of the compounds of formula (I) to inhibit the metalloproteases MMP-1, MMP-2, MMP-3, MMP-7, MMP-9, MMP-13 and MMP-14. The results obtained show that the compounds of the invention generally have IC₅₀ values for MMP-12 that are from 5 to more than 100 times lower than the IC₅₀ values obtained for the same compound with the other metalloproteases tested, thus proving their capacity for selective inhibition with respect to metalloprotease-12 (MMP-12). More specifically, the compounds of the present invention generally show selectivity with a factor of greater than 50 towards the metalloproteases mentioned above, except with regard to MMP-13. Thus, the compounds of the present invention also show inhibitory activity on MMP-13, also allowing the use of the pharmaceutical compositions containing one or more compounds of the invention for the treatment of pathologies associated with an activity of MMP-13. Among these pathologies that may be mentioned, as a guide and with no limitation, are cancer, osteoporosis, osteoarthritis, arthritis, rheumatoid arthritis, atherosclerosis, multiple sclerosis, cardiac insufficiency, asthma and chronic obstructive bronchopneumopathy.

By way of example and with no limitation of the invention, the table shows a number of results of activity of the compounds of the invention with respect to MMP-12 and MMP-13.

| | IC₅₀ (μM) | IC₅₀ (μM) |
|---|---|---|
| Example | MMP-12 | MMP-13 |
| 1 | 0.140 | 2.8 |
| 2 | 0.007 | 0.038 |
| 3 | 0.024 | 0.085 |

1. A compound of formula (I): 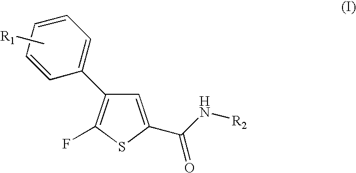
a steroisomer thereof, or a pharmaceutically acceptable salt of said compound or said steroisomer, wherein:
R₁ is halo(C₁-C₆)alkoxy, (C₁-C₆)alkyl, (C₁-C₆)alkoxy, (C₁-C₆)alkylthio or —R₃;
R₂ is -C₁-C₄)alkylenyl-R₄ wherein said (C₁-C₄)alkylenyl is optionally and independently substituted with carboxyl, carboxy(C₁-C₆)alkyl-, (C₁-C₆)alkyloxycarbonyl, (C₁-C₆)alkyloxycarbonyl(C₁-C₆)alkyl-, aminocarbonyl or aminocarbonyl(C₁-C₆)alkyl, wherein said amino from aminocarbonyl or aminocarbonyl(C₁-C₆)alkyl is optionally and independently substituted with one to two (C₁-C₆)alkyl;
R₃ is phenyl, cyclohexyl or heterocycle, optionally substituted with one to two substituents, independently selected from halogen, halo(C₁-C₆)alkyl-, halo(C₁-C₆)alkoxy-, (C₁-C₆)alkyl, cyano, cyano(C₁-C₆)alkyl-, hydroxyl, (C₁-C₆)alkoxy, phenoxy, (C₁-C₆)alkyl-SO₂—, (C₁-C₆)alkylcarbonyl, benzoyl, hydroxy(C₁-C₆)alkyl-, or amino, said amino optionally and independently substituted with one to two (C₁-C₆)alkyl;
R₄ is phenyl, cyclohexyl or morpholin-4-yl, optionally and independently substituted with one to two halogen, —OR₅, —CO₂R₅ or —W—CO₂R₅;
R₅ is hydrogen or (C₁-C₆)alkyl; and
W is (C₁-C₆)alkylenyl.

2. A compound of claim 1 wherein R₁ is located in the para position, or a stereoisomer thereof, or a pharmaceutically acceptable salt of said compound or said stereoisomer.

3. A compound of claim 1 wherein R₁ is trifluoromethoxy, 4-acetylphenyl, 4-pyridyl, 3-pyridyl, N-pyrrolidinyl, 1-methylpyrrol-3-yl, 3,6-dihydro-2H-pyrid-1-yl, 2-hydroxyphenyl or 2-hydroxy-4-pyridyl, or a stereoisomer thereof, or a pharmaceutically acceptable salt of said compound or said stereoisomer.

4. A compound of claim 3 wherein R₁ is trifluoromethoxy, 4-acetylphenyl or 4-pyridyl, or a stereoisomer thereof, or a pharmaceutically acceptable salt of said compound or said stereoisomer.

5. A compound of claim 1 wherein R₄ is cyclohexyl substituted in position 4- with a carboxyl group, or a stereoisomer thereof, or a pharmaceutically acceptable salt of said compound or said stereoisomer.

6. A compound of claim 5 wherein said carboxyl is trans, or a pharmaceutically acceptable salt of said compound.

7. A compound of claim 1 wherein R₂ is -methylenyl-R₄ wherein said methylenyl is substituted with carboxymethyl or aminocarbonylmethyl and wherein R₄ is phenyl, or a stereoisomer thereof, or a pharmaceutically acceptable salt of said compound or said stereoisomer.

8. A compound of claim 6 wherein the carbon atom bearing R₄ is (R) absolute configuration, or a pharmaceutically acceptable salt of said compound.

9. A compound selected from:
trans-4-({[5-fluoro-4-(4-trifluoromethoxyphenyl)thien-2-yl]carboxamido}methyl)cyclohexanecarboxylic acid;
trans-4-({[5-fluoro-4-(4-[4-acetylphenyl]phenyl)thien-2-yl]carboxamido}methyl)cyclohexanecarboxylic acid;
3-(R)-phenyl-3-({5-fluoro-4-[4-(4-acetylphenyl)-phenyl]thien-2-yl}carboxamido)propanoic acid;
3-(R)-phenyl-3-({5-fluoro-4-[4-trifluoromethoxyphenyl]thien-2-yl}carboxamido)propanoic acid;
3-(R)-phenyl-3-({5-fluoro-4-[4-(4-acetylphenyl)phenyl]thien-2-yl}carboxamido)propanamide; or
3-(R)-phenyl-3-({5-fluoro-4-[4-(4-pyridyl)-phenyl]thien-2-yl}carboxamido)propanoic acid hydrochloride; or
a stereoisomer thereof, or a pharmaceutically acceptable salt of said compound or said stereoisomer.

10. A pharmaceutical composition comprising a compound of claim 1, a stereoisomer thereof, or a pharmaceutically acceptable salt of said compound or stereoisomer, and a pharmaceutically acceptable carrier, vehicle or dilluent.

11. A method of treating pathologies requiring the action of a metalloprotease-12 or a metalloprotease-13 inhibitor in a mammal comprising administering to said mammal in need of such treatment a therapeutically effective amount of a compound of claim 1, a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.

12. A method of claim 11 wherein said pathology requires the action of a metalloprotease-12 inhibitor.

13. A method of treating chronic obstructive bronchopneumopathy (COPD), emphysema, chronic bronchitis, chronic pulmonary inflammation, asthma, mucoviscidosis, acute respiratory distress syndrome (ARDS), respiratory allergies including allergic rhinitis, and also diseases associated with the production of TNFα, including severe fibrotic pulmonary disease, pulmonary sarcoidosis or silicosis in a mammal comprising administering to said mammal in need of such treatment a therapeutically effective amount of a compound of claim 1, or a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.

14. A method of treating cancer, osteoporosis, osteoarthritis, arthritis, rheumatoid arthritis, atherosclerosis, multiple sclerosis or cardiac insufficiency in a mammal comprising administering to said mammal in need of such treatment a therapeutically effective amount of a compound of claim 1, or a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.

15. A method of treating chronic obstructive bronchopneumopathy, emphysema or chronic bronchitis in a mammal comprising administering to said mammal in need of such treatment a therapeutically effective amount of a compound of claim 1, or a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.

16. A method of treating tobacco-related emphysema in a mammal comprising administering to said mammal in need of such treatment a therapeutically effective amount of a compound of claim 1, or a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.

17. A method of treating asthma in a mammal comprising administering to said mammal in need of such treatment a therapeutically effective amount of a compound of claim 1, or a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.
